(19) United States
(12) Patent Application Publication
(10) Pub. No.: US 2014/0168398 A1
(43) Pub. Date: Jun. 19, 2014

(54) BODY FLUID IMAGING SYSTEM AND METHOD, AND DEPTH OF FIELD EXTENSION IMAGING DEVICE

(71) Applicants: Xin Guo, Shanghai (CN); Zhao Hui Du, Shanghai (CN)

(72) Inventors: Xin Guo, Shanghai (CN); Zhao Hui Du, Shanghai (CN)

(73) Assignee: SIEMENS HEALTHCARE DIAGNOSTICS INC., Tarrytown, NY (US)

(21) Appl. No.: 14/008,131

(22) PCT Filed: Mar. 28, 2012
(86) PCT No.: PCT/US12/30900

§ 371 Date: Jan. 29, 2014

(30) Foreign Application Priority Data
Mar. 31, 2011 (CN) 201110080747.9

(51) Int. Cl. A61B 5/00

(52) U.S. Cl. CPC A61B 5/0033
USPC 348/77

(57) ABSTRACT

Disclosed in the present invention is a body fluid imaging system, a body fluid imaging method and a depth of field extension imaging device. The depth of field extension imaging device includes: an interface unit for receiving the light refracted and/or reflected by the body fluid sample in a current body fluid sample container; and a depth of field extension unit for carrying out wavefront coding and convergence processing on the light received by said interface unit. By means of the system, method and device of the present invention, the time needed by the imaging process can be reduced, thus improving the productivity of the system; and error generated due to frequently adjusting the relative position between the body fluid sample container, the depth of field extension imaging device and the image sensor can be avoided, thus improving the reliability of the system.

### PRIORITY STATEMENT

This application claims benefit under 35 U.S.C. §119 of Chinese Patent Application Number CN 201110080747.9 filed Mar. 31, 2011, the entire contents of which are hereby incorporated herein by reference.

### TECHNICAL FIELD

The present invention relates to imaging technology and, particularly, to a body fluid imaging system and method, and a depth of field extension imaging device.

### BACKGROUND OF THE INVENTION

Body fluid imaging is one of the most useful techniques for biological research and clinical applications. In general, body fluid is a liquid mixture, including tiny shaped ingredients of various shapes. Body fluid includes: blood, saliva, urine, spermatic fluid, cerebrospinal fluid, tear, sweat, etc. By way of forming an image for the shaped ingredients in the body fluid sample, the obtained clear image can provide valuable information for relevant biological research, clinical diagnostics, pathology, etc.

In general, a microscope can be used to form an image for the shaped ingredients in the body fluid sample. Taking a urine sample as an example, the operation is roughly as follows: inject the urine sample into a urine sample container, and use a microscope having a digital image sensor to capture a bright field image of the shaped ingredients in the urine sample. Currently, theoretical research is mainly focused on the latest technology which is capable of improving image quality and resolving power, however, clinical applications prefer fully automatic systems which can take speed, accuracy, reliability and costs into account.

Currently, with the development of mechanical automation and image recognition technology, the fully automatic clinical body fluid inspection technique is increasingly generalized and applied. The application of the following three techniques promotes the development of fully automatic clinical body fluid inspection technology: 1. Auto fluid system, which system is capable of precisely collecting, assigning and discarding the body fluid sample; 2. Microscope having an auto-focusing optimization function, which is capable of capturing a digital image of the body fluid sample; 3. Image recognition software, which software is capable of processing the digital image captured by the microscope, so as to classify and count various shaped ingredients in the body fluid sample. These three techniques can be combined to design a system, and this system can be used to automatically generate an inspection report making significant sense for clinical diagnosis.

The existing static microscope technique is widely used in practical applications, such as clinical body fluid inspection. Such a static microscope system will be introduced hereinafter by way of a particular example. FIG. 1 is a structural schematic view of a static microscope system in the prior art. This system includes: a light source 10, a body fluid sample container 11, an imaging element 12, an image sensor 13 and an image processor 14.

Among them, the light source 10 illuminates the body fluid sample from the bottom or top of the body fluid sample container 11. The imaging element 12 is used to receive the light beam refracted and/or reflected by the body fluid sample, and carry out convergence processing on the received light beam. The digital image sensor 13 is used to receive the light beam converged by said imaging element 12, form an image for the shaped ingredients in said body fluid sample, record the formed image, and send the image to said image processor 14. Said image processor 14 is used to carry out further processing on said image.

In this case, the digital image sensor 13 can be a charge coupling device (CCD), a complementary metal-oxide semiconductor (CMOS), or other digital imaging devices capable of forming an image. The imaging element 12 can be a glass lens, a polymer lens, a liquid lens, an electromagnetic filter, a Fresnel zone plate, a photonic crystal, etc.

If it is necessary to obtain a clear image, the following condition should be satisfied:

1/do+1/di−1/f=0, wherein do represents the distance between the shaped ingredients and the imaging element 12, di represents the distance between the imaging element 12 and the image sensor 13, and f represents the focus length of the imaging element 12.

Depth of field refers to a distance range within which a clear image of the shaped ingredients can be acquired in the axial direction of the imaging system. In this embodiment, the shaped ingredients within this distance range (or DOF) can all be clearly imaged on the image sensor 13. In general, the depth of field (DOF) of the imaging element 12 is very small, generally only 2-10 micrometers. If the thickness of the cavity of the body fluid sample container 11 is much greater than the depth of focus, in order to enable all the shaped ingredients in the body fluid sample to be clearly imaged, it is necessary to adjust the best focus plane of the imaging element 12 to the bottom of the body fluid sample container 11, until all the shaped ingredients in the body fluid sample have subsided to the bottom of the cavity, and then form an image for all the shaped ingredients at the bottom of the cavity. Alternatively, in order to enable all the shaped ingredients in the body fluid sample to be clearly imaged, it is necessary to effectively regulate do and di so as to obtain a clear image, and it is necessary to uninterruptedly adjust the relative distances between the body fluid sample container 11, the imaging element 12 and the image sensor 13 during the imaging.

In order to obtain a clear image of all shaped ingredients in the entire body fluid sample, it is necessary to wait until all shaped ingredients in the body fluid sample have subsided to the bottom of the body fluid sample container 11, which to some extent extends the processing duration for each body fluid sample, resulting in the system having low productivity; or, it is necessary to carry out frequent mechanical adjustment on the lens during imaging, so as to effectively regulate the relative positions between the body fluid sample container 11, the imaging element 12 and the image sensor 13 to obtain the clear image of all the shaped ingredients in the entire body fluid sample. However, mechanical adjustment will introduce errors which decreases the reliability of the system.

### SUMMARY OF THE INVENTION

In view of this, the embodiments of the present invention provide a body fluid imaging system and method and a depth of field extension imaging device to improve the imaging productivity of the body fluid sample and improve the reliability of the system.

The embodiments of the present invention provide a depth of field extension imaging device, which device includes:

an interface unit for receiving the light refracted and/or reflected by a body fluid sample in a current body fluid sample container; and

a depth of field extension unit for carrying out wavefront coding and convergence processing on the light received by said interface unit.

In a preferred embodiment of the present invention, the depth of field extension unit includes:

a depth of field extension element for carrying out wavefront coding on the light received by said interface unit; and

an imaging element for receiving the coded light from said depth of field extension element, and carrying out convergence processing on the coded light; or

it includes:

an imaging element for carrying out convergence processing on the light received by said interface unit; and

a depth of field extension element for receiving the convergence processed light from said imaging element, and carrying out wavefront coding on the converged light.

Another embodiment of the present invention provides a body fluid imaging system, said system including:

a light source for illuminating a body fluid sample in a current body fluid sample container;

a depth of field extension imaging device as mentioned above; and

an image sensor for receiving the wavefront coded and converged light from said depth of field extension imaging device, and forming an image for the shaped ingredients in the body fluid sample according to said wavefront coded and convergence processed light.

In a preferred embodiment of the present invention, said system further includes:

an image decoder for decoding the image formed by said image sensor, so as to obtain a clearly focused image.

In a preferred embodiment of the present invention, said system further includes:

an image processor for identifying and counting the shaped ingredients in the clearly focused image decoded by said image decoder.

In a preferred embodiment of the present invention, said system further includes:

an image processor for receiving the image formed for the shaped ingredients from said image sensor, and identifying and counting the shaped ingredients in said image.

In a preferred embodiment of the present invention, said system further includes:

a channel control device for determining that the body fluid sample in the current body fluid sample container is the first body fluid sample of n body fluid samples, for determining, after said image sensor has formed an image for the shaped ingredients in said first body fluid sample, the second body fluid sample, and so forth, until said image sensor has completed forming an image for the shaped ingredients in the (n−1)th body fluid sample, and for determining the nth body fluid sample, wherein n is a natural number greater than 1.

In a preferred embodiment of the present invention, said body fluid sample in the current body fluid sample container is the first body fluid sample of n body fluid samples, wherein n is a natural number greater than 1, and the system further includes:

a channel control device for controlling said light source to illuminate said first body fluid sample, for controlling said depth of field extension imaging device to receive the light refracted and/or reflected by said first body fluid sample, for carrying out coding and convergence processing on the received light, for controlling, after said image sensor has formed an image for the shaped ingredients in said first body fluid sample, said light source to illuminate the second body fluid sample, for controlling said depth of field extension imaging device to receive the light refracted and/or reflected by said second body fluid sample, for carrying out coding and convergence processing on the received light, and so forth, until the images for the shaped ingredients in the n body fluid samples have been completely formed.

Another embodiment of the present invention provides a body fluid imaging method, the method including:

receiving the light refracted and/or reflected by a current body fluid sample;

carrying out wavefront coding and convergence processing on the received light;

according to said wavefront coded and converged light, forming an image for the shaped ingredients in the current body fluid sample.

In a preferred embodiment of the present invention, the method further includes:

decoding the image formed for the current body fluid sample, so as to obtain a clearly focused image.

In a preferred embodiment of the present invention, the method further includes:

identifying and counting the shaped ingredients in said image formed for the current body fluid sample.

Another embodiment of the present invention provides a computer program product, which computer program product includes computer program codes, and when a computer unit executes the computer program codes, the steps of the above-described method will be performed.

Another embodiment of the present invention provides a readable electronic storage medium used to store the above-mentioned computer program codes.

It can be seen from the above-described solution that in the embodiments of the present invention, a depth of field extension device is added into the microscope imaging system, which depth of field extension device carries out wavefront coding processing on the light projected thereon, thereby enlarging the depth of field of the imaging system. Thus, during the imaging processing of the body fluid sample, it is unnecessary to wait until the substances in the body fluid sample have subsided to the bottom of body fluid sample container and then form an image for the body fluid sample, thereby reducing the processing duration for the entire imaging process, and improving the productivity of the system. In addition, because the depth of field is enlarged, an image for all the substances in the body fluid sample can be formed without needing to frequently adjust the relative positions between the body fluid sample container, the depth of field extension device and the image sensor, thereby reducing error resulting from mechanical adjustment, and improving the reliability of the system.

### BRIEF DESCRIPTION OF THE DRAWINGS

FIG. 1 is a structural schematic view of a static microscope system in the prior art;

FIG. 2 is a structural schematic view of a first preferred embodiment of a body fluid imaging system according to the present invention;

FIG. 2(a) is a structural schematic view of a first preferred embodiment of a depth of field extension imaging device according to the present invention;

FIG. 2(b) is a structural schematic view of a second preferred embodiment of the depth of field extension imaging device according to the present invention;

FIG. 3 is a structural schematic view of a second preferred embodiment of the body fluid imaging system according to the present invention;

FIG. 4(a) is a structural schematic view of a third preferred embodiment of the body fluid imaging system according to the present invention;

FIG. 4(b) is a structural schematic view of a fourth preferred embodiment of the body fluid imaging system according to the present invention;

FIG. 5 is a structural schematic view of a first preferred embodiment of a multi-channel body fluid imaging system according to the present invention;

FIG. 6 is a structural schematic view of a second preferred embodiment of the multi-channel body fluid imaging system according to the present invention;

FIG. 7 is a schematic flow chart of a first preferred embodiment of the body fluid imaging method according to the present invention; and

FIG. 8 is a schematic flow chart of a second preferred embodiment of the body fluid imaging method according to the present invention.

### DETAILED DESCRIPTION OF THE DRAWINGS

10-light source 11-body fluid sample container 12-imaging element 13-image sensor 14-image processor

20-light source 21-body fluid sample container 22-depth of field extension imaging device 23-image sensor

2210a-interface unit 2220a-depth of field extension unit 2221a-depth of field extension element 2222a-imaging element

2210b-interface unit 2220b-depth of field extension unit 2221b-imaging element 2222b-depth of field extension element

30-light source 31-body fluid sample container 32-depth of field extension imaging device 33-image sensor 34-image decoder

40a-light source 41a-body fluid sample container 42a-depth of field extension imaging device 43a-image sensor 44a-image processor

40b-light source 41b-body fluid sample container 42b-depth of field extension imaging device 43b-image sensor 44b-image decoder 45b-image processor

50-light source 51-channel control device 52(1) 52(2) 52(n)-body fluid sample container 53-depth of field extension imaging device 54-image sensor

60-light source 61-channel control device 62(1) 62(2) 62(n)-body fluid sample container 63-depth of field extension imaging device 64-image sensor

701-carry out wavefront coding and convergence processing on the light 702-form an image for the shaped ingredients in the body fluid

801-the light source illuminates the body fluid sample n 802-carry out wavefront coding and convergence processing on the light 803-form an image for the shaped ingredients 804-decode the formed image 805-identify and count the shaped ingredients in the image 806-judge whether there is any body fluid samples not yet inspected

### Particular Embodiments

In order to make the objects, technical solutions and advantages of the present invention more apparent, the present invention will be further described in detail below with reference to the accompanying drawings and embodiments.

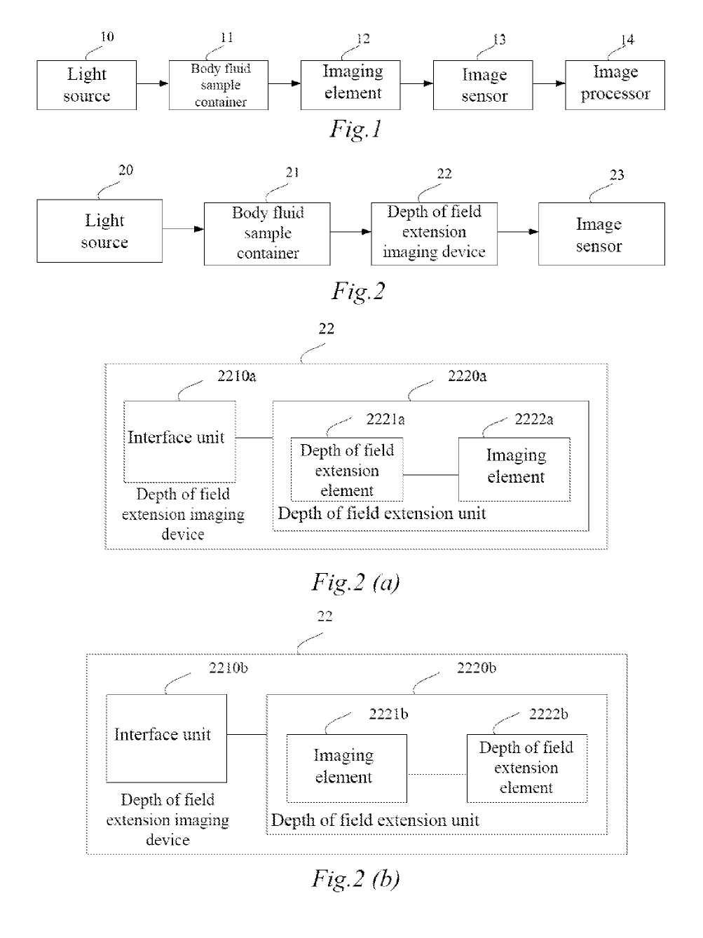

FIG. 2 is a structural schematic view of a first preferred embodiment of a body fluid imaging system according to the present invention. In the embodiment of the present invention, the body fluid can include: blood, saliva, urine, spermatic fluid, cerebrospinal fluid, tear, sweat, etc.

The system shown in FIG. 2 includes: a light source 20, a body fluid sample container 21, a depth of field extension imaging device 22, and an image sensor 23. The light source 20 is used to emit light of a certain wavelength, to illuminate the body fluid sample in the body fluid sample container 21 from the bottom or top of the body fluid sample container 21. The depth of field extension imaging device 22 is used to receive the light refracted and/or reflected by the body fluid sample, and carry out wavefront coding and convergence processing on the received light. The image sensor 23 is used to receive the wavefront coded and converged light from the depth of field extension imaging device 22, form an image for the shaped ingredients in the body fluid sample according to the wavefront coded and converged light, and record the formed image.

FIG. 2(a) is a structural schematic view of a first preferred embodiment of a depth of field imaging extension device according to the present invention. In this embodiment, the depth of field imaging extension device 22 includes: an interface unit 2210a and a depth of field extension unit 2220a. Among them, the depth of field extension unit 2220a includes a depth of field extension element 2221a and an imaging element 2222a. In this case, the interface unit 2210a is used to receive the light refracted and/or reflected by the body fluid sample. The depth of field imaging extension element 2221a is used to carry out wavefront coding on the received light, and output the wavefront coded light to the imaging element 2222a. The imaging element 2222a carries out convergence processing on the wavefront coded light.

FIG. 2(b) is a structural schematic view of a second preferred embodiment of the depth of field extension imaging device according to the present invention. In this embodiment, the depth of field imaging extension device 22 includes: an interface unit 2210b and a depth of field extension unit 2220b. In this case, the depth of field extension unit 2220b includes an imaging element 2221b and a depth of field extension element 2222b. In this case, the interface unit 2210b is used to receive the light refracted and/or reflected by the body fluid sample. The imaging element 2221b is used to carry out convergence processing on the received light, and output the converged light to the depth of field extension element 2222b. The depth of field extension element 2222b is used to carry out wavefront coding on the light converged by the imaging element 2221b.

FIG. 3 is a structural schematic view of a second preferred embodiment of the body fluid imaging system according to the present invention. As compared to the embodiment shown in FIG. 2, the body fluid imaging system shown in this embodiment further includes an image decoder 34 in addition to including a light source 30, a body fluid sample container 31, a depth of field extension imaging device 32 and an image sensor 33. The image decoder 34 is used to decode the image formed by the image sensor 33, so as to obtain a clearly focused image that can be perceived by the human eye and by machine.

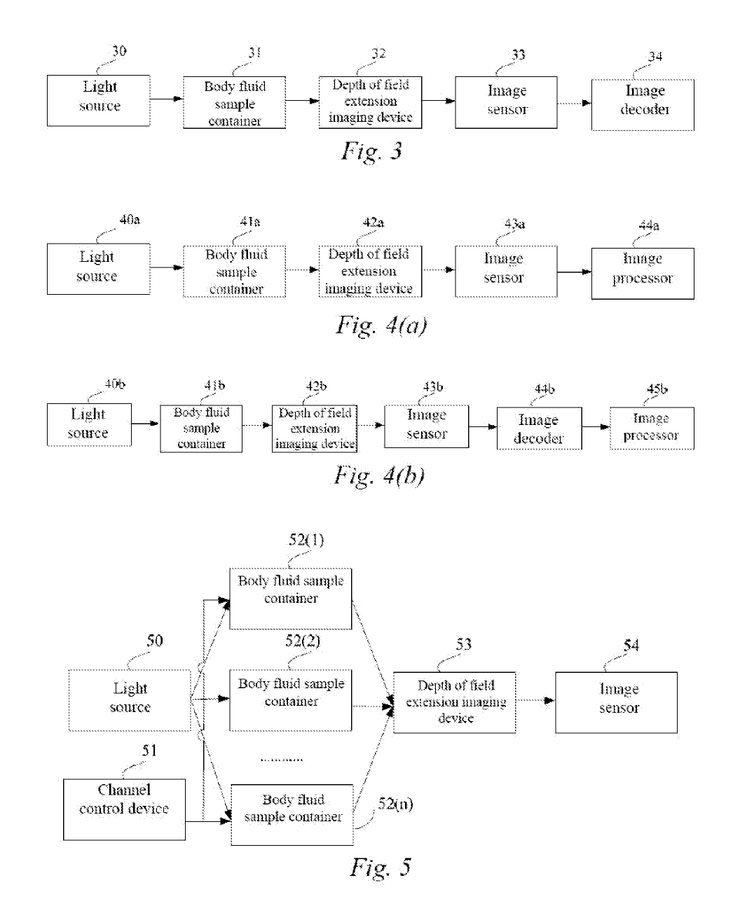

FIG. 4(a) is a structural schematic view of a third preferred embodiment of the body fluid imaging system according to the present invention. As compared to the embodiment shown in FIG. 2, the body fluid imaging system shown in this embodiment further includes an image processor 44a in addition to including a light source 40a, a body fluid sample container 41a, a depth of field extension imaging device 42a and an image sensor 43a. The image processor 44a is used to receive the image formed for the shaped ingredients by the image sensor 43a, and identify and count the shaped ingredients in the image.

FIG. 4(b) is a structural schematic view of a fourth preferred embodiment of the body fluid imaging system according to the present invention. As compared to the embodiment shown in FIG. 3, the body fluid imaging system shown in this embodiment further includes an image processor 45b in addition to including a light source 40b, a body fluid sample container 41b, a depth of field extension imaging device 42b and an image sensor 43b and an image decoder 44b. The image processor 45b is used to receive the decoded image from the image decoder 44b, and identify and count the shaped ingredients in the image.

FIG. 5 is a structural schematic view of a first preferred embodiment of a multi-channel body fluid imaging system according to the present invention. The system includes: a light source 50, a channel control device 51, a body fluid sample container 52(1), a body fluid sample container 52(2), . . . , a body fluid sample container 52(n), a depth of field extension imaging device 53, and an image sensor 54, wherein n is a natural number greater than 1. The channel control device 51 is used to determine the first body fluid sample among n body fluid samples, i.e. body fluid sample 1; said light source 50 illuminates the bottom or top of the body fluid sample container 52(1) containing the body fluid sample 1; said depth of field extension imaging device 53 receives the light refracted and/or reflected by the body fluid sample 1, and carries out wavefront coding and convergence processing on the light; said image sensor 54 receives the light coded and converged by the depth of field extension imaging device 53, forms an image for the shaped ingredients in the body fluid sample 1 according to the wavefront coded and converged light, and records the formed image. After the imaging of the body fluid sample 1 is finished, the channel control device 51 determines the second body fluid sample, i.e. body fluid sample 2; the light source 50, depth of field extension imaging device 53 and image sensor 54 perform the above-mentioned corresponding operation to carry out imaging processing on the second body fluid sample 2. The rest can be done in the same manner, after the image sensor 54 has formed an image for the shaped ingredients in the (n−1)th body fluid sample, the channel control device determines the nth body fluid sample. In this embodiment, an image decoder and an image processor can also be included simultaneously, or simply one of them can be included, and the functions of the image decoder and the image processor are the same as above.

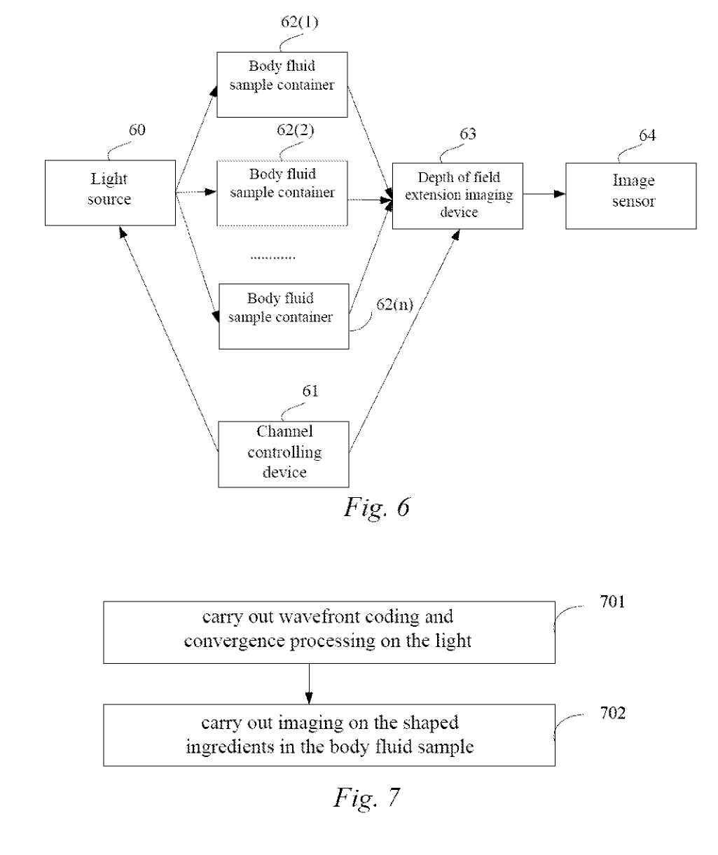

FIG. 6 is a structural schematic view of a second preferred embodiment of the multi-channel body fluid imaging system according to the present invention. The system includes: a light source 60, a channel control device 61, a body fluid sample container 62(1), a body fluid sample container 62(2), . . . , a body fluid sample container 62(n), a depth of field extension imaging device 63, and an image sensor 64, wherein n is a natural number greater than 1.

When the body fluid sample 1 is being inspected, the channel control device 61 controls the light source 60 to illuminate the bottom or the top of the body fluid sample container 62(1) containing the body fluid sample 1, controls the depth of field extension imaging device 63 to receive the light refracted and/or reflected by the body fluid sample 1, and carries out wavefront coding and convergence processing on the received light. After the imaging processing of the body fluid sample 1 is finished, the channel control device 61 controls the light source 60 to illuminate the body fluid sample 2, and controls the depth of field extension imaging device 63 to receive the light refracted and/or reflected by the body fluid sample 2, and carries out wavefront coding and convergence processing on the received light. In this embodiment, an image sensor 64 for receiving the light coded and converged by the depth of field extension imaging device 63 is also further included, for forming an image for the shaped ingredients in the body fluid sample 1 according to the wavefront coded and converged light, and for recording the formed image. The rest can be done in the same manner, until the imaging processing of the nth body fluid sample is finished. In this embodiment, an image decoder and an image processor can also be included simultaneously, or simply one of them can be included, and the functions of the image decoder and the image processor are the same as above.

In the multi-channel body fluid imaging systems according to the present invention as shown in FIGS. 5 and 6, each body fluid sample container together with the light source and the depth of field extension imaging device and so on forms a fluid channel, and n fluid channels are independent from each other and can be separately operated. For example, after the imaging processing of the body fluid sample 1 is finished, the imaging system can be switched to another fluid channel to perform imaging processing on another body fluid sample while cleaning the fluid channel 1 which forms an image of the body fluid sample 1. Thus, by way of parallel operation, the efficiency of the imaging system can be improved.

In the embodiments of the present invention, the imaging element can be a glass lens, a polymer lens, a liquid lens, an electromagnetic filter, a Fresnel zone plate, a photonic crystal, etc. The digital image sensor 23 can be a charge coupling device (CCD), a complementary metal-oxide semiconductor (CMOS), or other digital imaging devices capable of forming an image. The depth of field extension element is generally made of an optical material, such as glass, or plastic thin film with the transparency, thickness or refractive index thereof being changeable.

In the embodiments of the present invention, a description is given taking the case where the imaging element is a lens as an example. The depth of field extension element and the imaging element can be spatially separated, or can be integrated together. In the case of being separated, the distance between the depth of field extension element and the lens is very small, generally less than a few millimeters. In the case of being integrated, the depth of field extension element can be the embossment structure on the surface of the lens, for example, it can be a pattern composed of separated areas of various radius and changeable lens thickness; or the depth of field extension element can be a pattern composed of lens areas made of materials with different refractive indexes. When the material used in the depth of field extension element has a different refractive index from that of the lens, this material can be coated on the surface of the lens in the separated areas to form the pattern.

In a preferred case, the depth of field extension element is a phase mask plate, which affects only the phase and not the amplitude of the light when carrying out wavefront coding on the light, and the optical system including such a depth of field extension element is a highly efficient optical system, capable of extending the depth of field of the imaging element more effectively.

The depth of field extension element affects the optical system in the following ways: generally, when an optical system forms an image of an object, the best imaging plane is within a certain depth of field range. The image plane is susceptible to the object distance, that is to say, the depth of the best imaging plane of the optical system is small. The imaging quality of this optical system is evaluated by the point spread function (abbreviated as PSF) or optical transfer function (abbreviated as OTF) of the optical system. After a depth of field extension element is added therein, a mask plate having phase modulation is inserted into the optical path of a traditional optical system. After the mask plate is added, the light rays emitted in different directions from a point on the object plane no longer converge into one point in the conjugated focus plane thereof, but become a uniform fine light beam at the front and rear of the focus plane, that is, this special phase mask plate changes the original direction of the light rays in the traditional optical system, thereby coding the wavefront of the light wave, causing the PSF or OTF of the entire optical system to be unsusceptible to defocusing. Although the OTF of the optical system is dropped after the depth of field extension element is added, as long as the OTF contains no zero point, the image can be restored by using a digital image processing method. Then, by way of digital image processing techniques, the final clear image is obtained by decoding the intermediate blur image which is unsusceptible to defocusing, so as to achieve the object of increasing the depth of field of the traditional optical system.

An introduction is given taking the case where the depth of field extension element is a cubic phase modulation (Cubic-PM) mask plate as an example. When the Cubic-PM mask plate has the following surface type: P(x)=α(x³+y³), the expression of the optical transfer function (OTF) of the optical system that can be worked out according to the geometric optics principle is already independent from the defocused wave aberration coefficient. This indicates that: the Cubic-PM mask plate effectively reforms the OTF of the optical system, and makes it unsusceptible to defocusing. α represents the parameter for adjusting the depth of field. When α=0 and P(x)=1, it shows that no mask plate is used or that the mask plate is a transparent shadow shield. The depth of field extension power increases with the increasing of the absolute value of α. However, the contrast of the image will decrease with the increasing of α. Therefore, in practical applications, there is a limit to the increasing of the depth of field, and in the situation where the contrast requirement is satisfied, the depth of field of the system can be increased by increasing the absolute value of α.

In another aspect, although the OTF of the optical system is made unsusceptible to the depth of field after the Cubic-PM plate is added, as compared to the focused OTF of the traditional optical system, the amplitude thereof is decreased significantly, while the greater the α of the phase plate, the worse it drops. However, within the effective frequency band range, the OTF of the wavefront coding system has no zero point or close zero point, which means that the information of the original system beyond the depth of field is not lost, but merely coded in a certain way. For a depth of field extension system, the concept of a best image plane no longer exists, because in a large range, the image obtained by the system is homogeneously blurred, or coded.

What is described above are preferred embodiments of the system and device according to the present invention, and a particular implementation of the method according to the present invention will be described in detail hereinafter.

FIG. 7 is a schematic flow chart of a first preferred embodiment of a body fluid imaging method according to the present invention. This embodiment includes the following steps:

Step 701: the depth of field extension imaging device receives the light refracted and/or reflected by the current body fluid sample, and carries out wavefront coding and convergence processing on the light.

Step 702: The image sensor receives the light coded and converged by the depth of field extension imaging device, and forms an image for the shaped ingredients in the body fluid sample according to the wavefront coded and converged light.

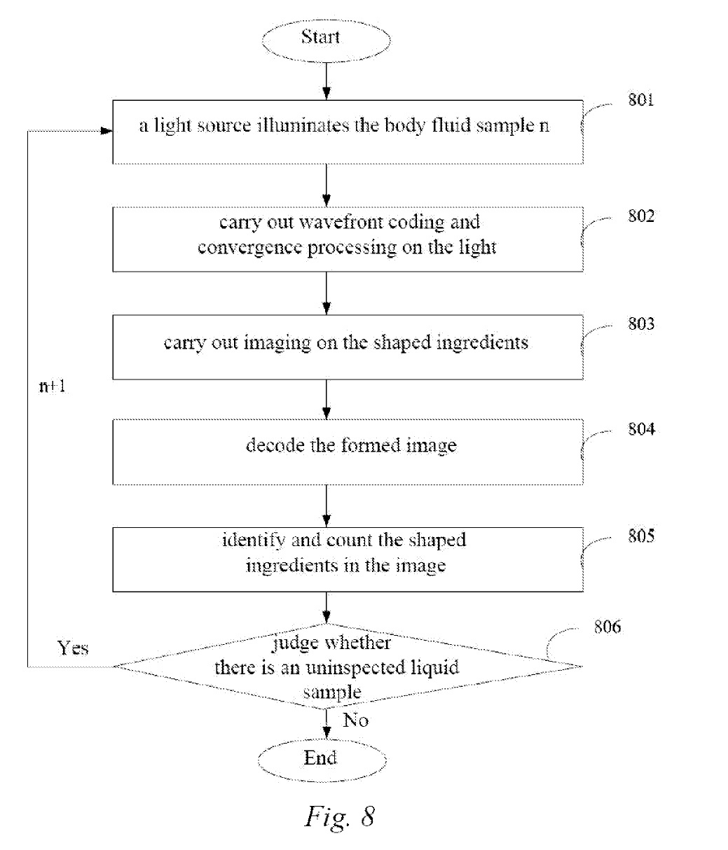

FIG. 8 is a schematic flow chart of a second preferred embodiment of the body fluid imaging method according to the present invention. This embodiment includes the following steps:

Step 801: the light source illuminates the body fluid sample n from the bottom or top of the body fluid sample container n. n is a natural number, for example, n=1 represents body fluid sample 1.

Step 802: the depth of field extension imaging device receives the light refracted and/or reflected by body fluid sample n, and carries out coding and convergence processing on the light.

Step 803: form an image for the shaped ingredients in the body fluid sample n according to the wavefront coded and convergence processed light, and record the formed image.

Step 804: decode the formed image to obtain the clearly focused image perceivable to the human eye or to a computer.

In the embodiments of the present invention, de-convolution processing can be carried out on the formed image to obtain the clearly focused image.

Step 805: receive the decoded image, identify and count the shaped ingredients in the image.

Step 806: judge whether there is a body fluid sample not yet inspected, if yes, return to step 801 to perform imaging on the next body fluid sample n+1, for example body fluid sample 2; otherwise end this process.

In this case, in step 802, it is feasible to firstly carry out wavefront coding on the refracted and/or reflected light, then to carry out convergence processing on the coded light, and it is also feasible to firstly carry out convergence on the refracted and/or reflected light, then to carry out wavefront coding on the converged light. According to the particular requirements, steps 804 and 805 or any one of them can also not be included.

The embodiments of the present invention utilize the depth of field extension device including a depth of field extension element to form an image for the body fluid sample, thereby extending the depth of field of microscope imaging, for example, the depth of field of the microscope can be extended to be 10-50 micrometers. Thus, during the imaging processing of the body fluid sample, it is not necessary to wait until the substances in the body fluid sample have subsided to the bottom of the body fluid sample container and then form an image for the body fluid sample, thereby reducing the processing duration for the entire imaging process, and improving the productivity of the system. In addition, because the depth of field is enlarged, the image for all the substances in the body fluid sample can be formed without needing to frequently adjust the relative positions between the body fluid sample container, the depth of field extension device and the image sensor, thereby reducing error resulting from mechanical adjustment, and improving the reliability of the system.

The present invention also provides a machine readable storage medium storing instructions for enabling a machine to execute the method for imaging a body fluid sample as described herein. Particularly, it is feasible to provide a system or device provided with the storage medium which is stored thereon with the software program codes for realizing the function of any one of the above-mentioned embodiments, and causing the computer (or CPU or MPU) of the system or device to read and execute the program codes stored in the storage medium.

In this case, the program codes per se read from the storage medium can realize the function of any one of the above-mentioned embodiments, therefore the program codes and the storage medium storing the same constitute a part of the present invention.

The embodiments of the storage medium for providing program codes can include floppy disc, hard disc, magnetic optical disc, optical disc (such as CD-ROM, CD-R, CD-RW, DVD-ROM, DVD-RAM, DVD-RW, DVD+RW), tape, nonvolatile storage card and ROM. Optionally, the program codes can be downloaded from the server computer via a communication network.

In addition, it should be clarified that the operating system running on the computer can be made to complete part of or all of the practical operations, not only by way of executing the program codes read by the computer, but also by means of instructions based on the program codes, thereby realizing the function of any one of the above-mentioned embodiments.

In addition, it can be understood that the program codes read from the storage medium are written to the memory provided in the extension board inserted inside the computer or to the memory provided in the extension unit connected to the computer, and subsequently the instructions based on the program codes cause the CPU mounted on the extension board or the extension unit to execute part of or all of the practical operations, thereby realizing the function of any one of the above-mentioned embodiments.

1. A depth of field extension imaging device, said device comprising:
an interface unit for receiving the light refracted and/or reflected by a body fluid sample in a current body fluid sample container, and
a depth of field extension unit for carrying out wavefront coding and convergence processing on the light received by said interface unit.

2. The device as claimed in claim 1, wherein said depth of field extension unit comprises:
a depth of field extension element for carrying out wavefront coding on the light received by said interface unit, and
an imaging element for receiving coding processed light from said depth of field extension element, and carrying out the convergence processing on the coded light; or
it comprises:
an imaging element for carrying out the convergence processing on the light received by said interface unit, and
a depth of field extension element for receiving the convergence processed light from said imaging element and carrying out the wavefront coding on the converged light.

3. A body fluid imaging system, said system comprising:
a light source for illuminating the body fluid sample in a current body fluid sample container,
a depth of field extension imaging device as claimed in claim 1, and
an image sensor for receiving the wavefront coded and converged light from said depth of field extension imaging device and forming an image of the shaped ingredients in the body fluid sample according to said wavefront coded and convergence processed light.

4. The body fluid imaging system as claimed in claim 3, wherein said system further comprises:
an image decoder for decoding the image formed by said image sensor, so as to obtain a clearly focused image.

5. The body fluid imaging system as claimed in claim 4, wherein said system further comprises:
an image processor for identifying and counting the shaped ingredients in the clearly focused image decoded by said image decoder.

6. The body fluid imaging system as claimed in claim 3, wherein said system further comprises:
an image processor for receiving the image formed for the shaped ingredients from said image sensor, and identifying and counting the shaped ingredients in said image.

7. The body fluid imaging system as claimed in claim 3, wherein said system further comprises:
a channel control device, which is used for determining that the body fluid sample in a current body fluid sample container is the first body fluid sample of n body fluid samples; for determining, after said image sensor has formed the image for the shaped ingredients in said first body fluid sample, the second body fluid sample, and so on, until said image sensor has completed forming images for the shaped ingredients in the (n−1)th body fluid sample; and for determining the nth body fluid sample, wherein n is a natural number greater than 1.

8. The body fluid imaging system as claimed in claim 3, wherein said body fluid sample in the current body fluid sample container is the first body fluid sample of n body fluid samples, wherein n is a natural number greater than 1, and the system further comprises:
a channel control device for controlling said light source to illuminate said first body fluid sample and for controlling said depth of field extension imaging device to receive the light refracted and/or reflected by said first body fluid sample, for carrying out the coding and convergence processing on the received light, for controlling, after said image sensor has formed the image for the shaped ingredients in said first body fluid sample, said light source to illuminate the second body fluid sample, and for controlling said depth of field extension imaging device to receive the light refracted and/or reflected by said second body fluid sample, for carrying out the coding and convergence processing on the received light, and so forth, until the imaging for the shaped ingredients in the nth body fluid sample has been completed.

9. A body fluid imaging method, said method comprising:
receiving the light refracted and/or reflected by a current body fluid sample;
carrying out wavefront coding and convergence processing on the received light; and
forming an image for the shaped ingredients in the current body fluid sample according to said wavefront coded and converged light.

10. The body fluid imaging method as claimed in claim 9, wherein the method further comprises:
decoding said image formed for the current body fluid sample, so as to obtain a clearly focused image.

11. The body fluid imaging method as claimed in claim 9, wherein the method further comprises:
identifying and counting the shaped ingredients in said image formed for the current body fluid sample.

12. A computer program product, the computer program product comprising computer program codes, so that when a computer unit executes the computer program codes, the steps as claimed in any one of claims 9 to 11 are executed.

13. A readable electronic storage medium, wherein the readable electronic storage medium is used to store the computer program codes as claimed in claim 12.
